(19) United States
(12) Patent Application Publication
(10) Pub. No.: US 2020/0181663 A1
(43) Pub. Date: Jun. 11, 2020

(54) COMBINED USE OF AT LEAST ONE ENDOPROTEASE AND AT LEAST ONE EXO-PROTEASE IN AN SSF PROCESS FOR IMPROVING ETHANOL YIELD

(71) Applicant: NOVOZYMES A/S, Bagsvaerd (DK)

(72) Inventor: Chee-Leong Soong, Raleigh, NC (US)

(73) Assignee: NOVOZYMES A/S, Bagsvaerd (DK)

(21) Appl. No.: 16/642,965

(22) PCT Filed: Aug. 28, 2018
(86) PCT No.: PCT/US2018/048238

§ 371 Date: Feb. 28, 2020

Related U.S. Application Data
(60) Provisional application No. 62/551,827, filed on Aug. 30, 2017.

(51) Int. Cl. C12P 19/14; C12P 19/02; C12P 7/06

(52) U.S. Cl. CPC C12P 19/14; C12P 7/06; C12P 19/02

(57) ABSTRACT

The present invention relates to improved processes for producing ethanol from starch-containing materials by the combined use of at least one endo-protease and at least one exo-protease in an SSF process, and wherein the endo-protease is selected from a family M35 endo-protease and the exo-protease is selected from a family S53 exo-protease. More particularly the exo-protease should make up at least 5% (w/w) of the protease mixture.

### REFERENCE TO SEQUENCE LISTING

This application contains a Sequence Listing in computer readable form. The computer readable form is incorporated herein by reference.

### FIELD OF THE INVENTION

The present invention relates to processes for producing fermentation products from gelatinized and/or un-gelatinized starch-containing material.

### BACKGROUND OF THE INVENTION

Production of fermentation products, such as ethanol, from starch-containing material is well-known in the art. Generally two different kinds of processes are used. The most commonly used process, often referred to as a “conventional process”, includes liquefying gelatinized starch at high temperature using typically a bacterial alpha-amylase, followed by simultaneous saccharification and fermentation carried out in the presence of a glucoamylase and a fermenting organism. Conventional starch-conversion processes, such as liquefaction and saccharification processes are described in, e.g., U.S. Pat. No. 3,912,590, EP252730 and EP063909.

Another well-known process, often referred to as a “raw starch hydrolysis”-process (RSH process) includes simultaneously saccharifying and fermenting granular starch below the initial gelatinization temperature typically in the presence of an acid fungal alpha-amylase and a glucoamylase.

U.S. Pat. No. 5,231,017-A discloses the use of an acid fungal protease during ethanol fermentation in a process comprising liquefying gelatinized starch with an alpha-amylase.

WO 2003/066826 discloses a raw starch hydrolysis process (RSH process) carried out on non-cooked mash in the presence of fungal glucoamylase, alpha-amylase and fungal protease.

WO 2007/145912 discloses a process for producing ethanol comprising contacting a slurry comprising granular starch obtained from plant material with an alpha-amylase capable of solubilizing granular starch at a pH of 3.5 to 7.0 and at a temperature below the starch gelatinization temperature for a period of 5 minutes to 24 hours; obtaining a substrate comprising greater than 20% glucose, and fermenting the substrate in the presence of a fermenting organism and starch hydrolyzing enzymes at a temperature between 10° C. and 40° C. for a period of 10 hours to 250 hours. Additional enzymes added during the contacting step may include protease.

WO 2010/008841 discloses processes for producing fermentation products, such as ethanol, from gelatinized as well as un-gelatinized starch-containing material by saccharifying the starch material using at least a glucoamylase and a metalloprotease and fermenting using a yeast organism. Particularly the metallo protease is derived form a strain of Thermoascus aurantiacus.

WO 2014/037438 discloses serine proteases derived from Meripilus giganteus, Trametes versicolor, and Dichomitus squalens and their use in animal feed.

WO 2015/078372 discloses serine proteases derived from Meripilus giganteus, Trametes versicolor, and Dichomitus squalens for use in a starch wet milling process.

WO 2003/048353 discloses a metalloprotease from Thermoascus aurantiacus.

WO 2013/102674 discloses exo-proteases belonging to family S53.

S53 proteases are known in the art, e.g., a S53 peptide from Grifola frondosa with accession number MER078639. A S53 protease from Postia placenta (Uniprot: B8PMI5) was isolated by Martinez et al in “Genome, transcriptome, and secretome analysis of wood decay fungus Postia placenta supports unique mechanisms of lignocellulose conversion”, 2009, Proc. Natl. Acad. Sci. USA 106:1954-1959.

Vanden Wymelenberg et al. have isolated a S53 protease (Uniprot: Q281W2) in “Computational analysis of the Phanerochaete chrysosporium v2.0 genome database and mass spectrometry identification of peptides in ligninolytic cultures reveal complex mixtures of secreted proteins”, 2006, Fungal Genet. Biol. 43:343-356. Another S53 polypeptide from Postia placenta (Uniprot:B8P431) has been identified by Martinez et al. in “Genome, transcriptome, and secretome analysis of wood decay fungus Postia placenta supports unique mechanisms of lignocellulose conversion”, 2009, Proc. Natl. Acad. Sci. U.S.A. 106:1954-1959.

Floudas et al have published the sequence of a S53 protease in “The Paleozoic origin of enzymatic lignin decomposition reconstructed from 31 fungal genomes”, 2012, Science, 336:1715-1719. Fernandez-Fueyo et al have published the sequences of three serine proteases in “Comparative genomics of Ceriporiopsis subvermispora and Phanerochaete chrysosporium provide insight into selective ligninolysis”, 2012, Proc Natl Acad Sci USA. 109:5458-5463 (Uniprot:M2QQ01, Uniprot:M2QWH2, Uniprot:M2RD67).

It is an object of the present invention to identify protease mixtures that will result in an increased ethanol yield in a starch to ethanol process, when said proteases are added/are present during saccharification and/or fermentation.

### SUMMARY OF THE INVENTION

The inventors of the present invention have surprisingly found that adding a mixture of endo-protease and exo-protease to the SSF process will result in an increased ethanol yield. The invention provides in a first aspect a process for producing a fermentation product from starch-containing material comprising:

a) saccharifying the starch-containing material at a temperature below the initial gelatinization temperature of said starch-containing material using a carbohydrate-source generating enzymes; and
b) fermenting using a fermenting organism; wherein
steps a) and/or b) is performed in the presence of an endo-protease and an exo-protease mixture, wherein the exo-protease makes up at least 5% (w/w) of the protease mixture on a total protease enzyme protein basis, and wherein the endo-protease is selected from a family M35 endo-protease and the exo-protease is selected from a family S53 exo-protease.

In a second aspect the invention provides a process for producing a fermentation product from starch-containing material comprising the steps of:

(a) liquefying starch-containing material at a temperature above the initial gelatinization temperature of said starch-containing material in the presence of an alpha-amylase;

(b) saccharifying the liquefied material obtained in step (a) using a carbohydrate-source generating enzyme;

(c) fermenting using a fermenting organism;

wherein steps b) and/or c) is performed in the presence of an endo-protease and an exo-protease mixture, wherein the exo-protease makes up at least 5% (w/w) of the protease mixture on a total protease enzyme protein basis, and wherein the endo-protease is selected from a family M35 endo-protease and the exo-protease is selected from a family S53 exo-protease.

In a third aspect, the invention relates to a composition comprising a mixture of endo-protease and exo-protease, wherein the endo-protease is selected from a family M35 endo-protease and the exo-protease is selected from a family S53 exo-protease.

### Definitions

Proteases: The term “protease” includes any enzyme belonging to the EC 3.4 enzyme group (including each of the eighteen subclasses thereof). The EC number refers to Enzyme Nomenclature 1992 from NC-IUBMB, Academic Press, San Diego, Calif., including supplements 1-5 published in 1994, Eur. J. Biochem. 223: 1-5; 1995, Eur. J. Biochem. 232: 1-6; 1996, Eur. J. Biochem. 237: 1-5; 1997, Eur. J. Biochem. 250: 1-6; and 1999, Eur. J. Biochem. 264: 610-650 respectively. The nomenclature is regularly supplemented and updated; see e.g. the World Wide Web (WWW) at http://www.chem.qmw.ac.uk/uibmb/enzyme/index.html.

Proteases are classified on the basis of their catalytic mechanism into the following groups: Serine proteases (S), Cysteine proteases (C), Aspartic proteases (A), Metalloproteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

Polypeptides having protease activity, or proteases, are sometimes also designated peptidases, proteinases, peptide hydrolases, or proteolytic enzymes. Proteases may be of the exo-type (exopeptidases) that hydrolyse peptides starting at either end thereof, or of the endotype that act internally in polypeptide chains (endopeptidases).

In particular embodiments, the proteases for use in the processes of the invention are selected from the group consisting of:
(a) proteases belonging to the EC 3.4.24 metalloendopeptidases;
(b) metalloproteases belonging to the M group of the above Handbook;
(c) metalloproteases not yet assigned to clans (designation: Clan MX), or belonging to either one of clans MA, MB, MC, MD, ME, MF, MG, MH (as defined at pp. 989-991 of the above Handbook);
(d) other families of metalloproteases (as defined at pp. 1448-1452 of the above Handbook);
(e) metalloproteases with a HEXXH motif;
(f) metalloproteases with an HEFTH motif;
(g) metalloproteases belonging to either one of families M3, M26, M27, M32, M34, M35, M36, M41, M43, or M47 (as defined at pp. 1448-1452 of the above Handbook); and
(h) metalloproteases belonging to family M35 (as defined at pp. 1492-1495 of the above Handbook).
   S53 protease: The term “S53” means a protease activity selected from:
   (a) proteases belonging to the EC 3.4.21 enzyme group; and/or
   (b) proteases belonging to the EC 3.4.14 enzyme group; and/or
   (c) Serine proteases of the peptidase family S53 that comprises two different types of peptidases: tripeptidyl aminopeptidases (exo-type) and endo-peptidases; as described in 1993, Biochem. J. 290:205-218 and in MEROPS protease database, release, 9.4 (31 Jan. 2011) (www.merops.ac.uk). The database is described in Rawlings, N. D., Barrett, A. J. and Bateman, A., 2010, “MEROPS: the peptidase database”, Nucl. Acids Res. 38: D227-D233.

For determining whether a given protease is a Serine protease, and a family S53 protease, reference is made to the above Handbook and the principles indicated therein. Such determination can be carried out for all types of proteases, be it naturally occurring or wild-type proteases; or genetically engineered or synthetic proteases.

The peptidases of the S53 family tend to be most active at acidic pH (unlike the homologous subtilisins), and this can be attributed to the functional importance of carboxylic residues, notably Asp in the oxyanion hole. The amino acid sequences are not closely similar to those in family S8 (i.e. serine endopeptidase subtilisins and homologues), and this, taken together with the quite different active site residues and the resulting lower pH for maximal activity, provides for a substantial difference to that family. Protein folding of the peptidase unit for members of this family resembles that of subtilisin, having the clan type SB.

S8 protease: Most members of this family are endopeptidases, and are active at neutral-mildly alkali pH. Many peptidases in the family are thermostable. Casein is often used as a protein substrate and a typical synthetic substrate is Suc-Ala-Ala-Pro-Phe-NHPhNO₂. Most members of the family are nonspecific peptidases with a preference to cleave after hydrophobic residues. Link to S10 family definition for activity and specificities: http://merops.sanger.ac.uk/cqi-bin/famsum?family=S8

S10 protease: The carboxypeptidases in family S10 show two main types of specificity. Some (e.g. carboxypeptidase C) show a preference for hydrophobic residues in positions P1 and P1″. Carboxypeptidases of the second set (e.g. carboxypeptidase D) display a preference for the basic amino acids either side of the scissile bond, but are also able to cleave peptides with hydrophobic residues in these positions. Link to S10 family definition for activity and specificities: http://merops.sanger.ac.uk/dgi-bin/famsum?family=S10

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has serine protease activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having serine protease activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Protease activity: The term “protease activity” means proteolytic activity (EC 3.4). There are several protease activity types such as trypsin-like proteases cleaving at the carboxyterminal side of Arg and Lys residues and chymotrypsin-like proteases cleaving at the carboxyterminal side of hydrophobic amino acid residues.

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 2, 3, 4, 5, 6, 7, 8, 9, 10, 11, or 12. Examples of assay-temperatures are 15, 20, 25, 30, 35, 37, 40, 45, 50, 55, 60, 65, 70, 80, 90, or 95° C. Examples of general protease substrates are casein, bovine serum albumin and haemoglobin. In the classical Anson and Mirsky method, denatured haemoglobin is used as substrate and after the assay incubation with the protease in question, the amount of trichloroacetic acid soluble haemoglobin is determined as a measurement of protease activity (Anson, M. L. and Mirsky, A. E., 1932, J. Gen. Physiol. 16: 59 and Anson, M. L., 1938, J. Gen. Physiol. 22: 79).

For the purpose of the present invention, protease activity may be determined using assays which are described in “Materials and Methods”, such as the Kinetic Suc-AAPF-pNA assay, Protazyme AK assay, Kinetic Suc-AAPX-pNA assay and o-Phthaldialdehyde (OPA). For the Protazyme AK assay, insoluble Protazyme AK (Azurine-Crosslinked Casein) substrate liberates a blue colour when incubated with the protease and the colour is determined as a measurement of protease activity. For the Suc-AAPF-pNA assay, the colourless Suc-AAPF-pNA substrate liberates yellow paranitroaniline when incubated with the protease and the yellow colour is determined as a measurement of protease activity.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having protease activity.

Variant: The term “variant” means a polypeptide having protease activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

The present invention relates to improved processes for producing ethanol from starch-containing materials by the combined use of at least one endo-protease and at least one exo-protease in an SSF process. More particularly the exo-protease should make up at least 5% (w/w) of the protease mixture on a total protease enzyme protein basis.

More specifically the present disclosure relates to a process for producing a fermentation product from starch-containing material comprising:

a) saccharifying the starch-containing material at a temperature below the initial gelatinization temperature of said starch-containing material using a carbohydrate-source generating enzymes; and
b) fermenting using a fermenting organism; wherein
steps a) and/or b) is performed in the presence of an endo-protease and an exo-protease mixture, and wherein the exo-protease makes up at least 5% (w/w) of the protease mixture on a total protease enzyme protein basis.

In a second aspect, the disclosure provides a process for producing a fermentation product from starch-containing material comprising the steps of:

(a) liquefying starch-containing material at a temperature above the initial gelatinization temperature of said starch-containing material in the presence of an alpha-amylase;

(b) saccharifying the liquefied material obtained in step (a) using a carbohydrate-source generating enzyme;

(c) fermenting using a fermenting organism;

wherein steps b) and/or c) is performed in the presence of an endo-protease and an exo-protease mixture, and wherein the exo-protease makes up at least 5% (w/w) of the protease mixture on a total protease enzyme protein basis.

Processes for producing fermentation products, e.g., ethanol, from starch-containing materials are generally well known in the art. Generally two different kinds of processes are used. The most commonly used process, often referred to as a “conventional process”, includes liquefying gelatinized starch at high temperature using typically a bacterial alpha-amylase, followed by simultaneous saccharification and fermentation carried out in the presence of a glucoamylase and a fermenting organism. Another well-known process, often referred to as a “raw starch hydrolysis”-process (RSH process) includes simultaneously saccharifying and fermenting granular starch below the initial gelatinization temperature typically in the presence of an acid fungal alpha-amylase and a glucoamylase.

Native starch consists of microscopic granules, which are insoluble in water at room temperature. When aqueous starch slurry is heated, the granules swell and eventually burst, dispersing the starch molecules into the solution. At temperatures up to about 50° C. to 75° C. the swelling may be reversible. However, with higher temperatures an irreversible swelling called “gelatinization” begins. During this “gelatinization” process there is a dramatic increase in viscosity. Granular starch to be processed may be a highly refined starch quality, preferably at least 90%, at least 95%, at least 97% or at least 99.5% pure or it may be a more crude starch-containing materials comprising (e.g., milled) whole grains including non-starch fractions such as germ residues and fibers. The raw material, such as whole grains, may be reduced in particle size, e.g., by milling, in order to open up the structure and allowing for further processing. In dry milling whole kernels are milled and used. Wet milling gives a good separation of germ and meal (starch granules and protein) and is often applied at locations where the starch hydrolysate is used in the production of, e.g., syrups. Both dry and wet milling is well known in the art of starch processing and may be used in a process of the invention. Methods for reducing the particle size of the starch containing material are well known to those skilled in the art.

As the solids level is 30-40% in a typical industrial process, the starch has to be thinned or “liquefied” so that it can be suitably processed. This reduction in viscosity is primarily attained by enzymatic degradation in current commercial practice.

Liquefaction is carried out in the presence of an alpha-amylase, preferably a bacterial alpha-amylase and/or acid fungal alpha-amylase. In an embodiment, a phytase is also present during liquefaction. In an embodiment, viscosity reducing enzymes such as a xylanase and/or beta-glucanase is also present during liquefaction.

During liquefaction, the long-chained starch is degraded into branched and linear shorter units (maltodextrins) by an alpha-amylase. Liquefaction may be carried out as a three-step hot slurry process. The slurry is heated to between 60-95° C. (e.g., 70-90° C., such as 77-86° C., 80-85° C., 83-85° C.) and an alpha-amylase is added to initiate liquefaction (thinning).

The slurry may in an embodiment be jet-cooked at between 95-140° C., e.g., 105-125° C., for about 1-15 minutes, e.g., about 3-10 minutes, especially around 5 minutes. The slurry is then cooled to 60-95° C. and more alpha-amylase is added to obtain final hydrolysis (secondary liquefaction). The jet-cooking process is carried out at pH 4.5-6.5, typically at a pH between 5 and 6. The alpha-amylase may be added as a single dose, e.g., before jet cooking.

The liquefaction process is carried out at between 70-95° C., such as 80-90° C., such as around 85° C., for about 10 minutes to 5 hours, typically for 1-2 hours. The pH is between 4 and 7, such as between 4.5 and 5.5. In order to ensure optimal enzyme stability under these conditions, calcium may optionally be added (to provide 1-60 ppm free calcium ions, such as about 40 ppm free calcium ions). After such treatment, the liquefied starch will typically have a “dextrose equivalent” (DE) of 10-15.

Generally liquefaction and liquefaction conditions are well known in the art.

Alpha-amylases for use in liquefaction are preferably bacterial acid stable alpha-amylases. Particularly the alpha-amylase is from an Exiguobacterium sp. or a Bacillus sp. such as e.g., Bacillus stearothermophilus or Bacillus licheniformis.

Saccharification may be carried out using conditions well-known in the art with a carbohydrate-source generating enzyme, in particular a glucoamylase, or a beta-amylase and optionally a debranching enzyme, such as an isoamylase or a pullulanase. For instance, a full saccharification step may last from about 24 to about 72 hours. However, it is common to do a pre-saccharification of typically 40-90 minutes at a temperature between 30-65° C., typically about 60° C., followed by complete saccharification during fermentation in a simultaneous saccharification and fermentation (SSF) process. Saccharification is typically carried out at a temperature in the range of 20-75° C., e.g., 25-65° C. and 40-70° C., typically around 60° C., and at a pH between about 4 and 5, normally at about pH 4.5.

The saccharification and fermentation steps may be carried out either sequentially or simultaneously. In an embodiment, saccharification and fermentation are performed simultaneously (referred to as “SSF”). However, it is common to perform a pre-saccharification step for about 30 minutes to 2 hours (e.g., 30 to 90 minutes) at a temperature of 30 to 65° C., typically around 60° C. which is followed by a complete saccharification during fermentation referred to as simultaneous saccharification and fermentation (SSF). The pH is usually between 4.2-4.8, e.g., pH 4.5. In a simultaneous saccharification and fermentation (SSF) process, there is no holding stage for saccharification, rather, the yeast and enzymes are added together and the process is then carried out at a temperature of 25-40° C., such as between 28° C. and 35° C., such as between 30° C. and 34° C., such as around 32° C. The SSF-process may be carried out at a pH from about 3 and 7, preferably from pH 4.0 to 6.5, or more preferably from pH 4.5 to 5.5.

In an embodiment fermentation is ongoing for 6 to 120 hours, in particular 24 to 96 hours.

Instead of the conventional process described above, the fermentation product, e.g., ethanol, may be produced from starch-containing material without gelatinization (i.e., without cooking) of the starch-containing material (often referred to as a “raw starch hydrolysis” process). The fermentation product, such as ethanol, can be produced without liquefying the aqueous slurry containing the starch-containing material and water. In one embodiment the process includes saccharifying (e.g., milled) starch-containing material, e.g., granular starch, below the initial gelatinization temperature, preferably in the presence of alpha-amylase and/or carbohydrate-source generating enzyme(s) to produce sugars that can be fermented into the fermentation product by a suitable fermenting organism. In this embodiment the desired fermentation product, e.g., ethanol, is produced from un-gelatinized (i.e., uncooked), preferably milled, cereal grains, such as corn.

Accordingly, in this aspect the invention relates to processes for producing a fermentation product from starch-containing material comprising the steps of:

a) saccharifying the starch-containing material at a temperature below the initial gelatinization temperature of said starch-containing material using a carbohydrate-source generating enzymes; and

b) fermenting using a fermenting organism; wherein steps a) and/or b) is performed in the presence of an endo-protease and an exo-protease mixture, and wherein the exo-protease makes up at least 5% (w/w) of the protease mixture.

In a particular embodiment steps a) and b) are performed simultaneously, wherein the saccharifying enzymes and fermenting organisms (e.g., yeast) are added together and then carried out at a temperature of 25-40° C. The SSF-process may be carried out at a pH from about 3 and 7, preferably from pH 4.0 to 6.5, or more preferably from pH 4.5 to 5.5. In an embodiment fermentation is ongoing for 6 to 120 hours, in particular 24 to 96 hours.

The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material may be determined as the temperature at which birefringence is lost in 5% of the starch granules using the method described by Gorinstein and Lii, 1992, Starch/Starke 44(12): 461-466. In one embodiment a temperature below the initial gelatinization temperature means that the temperature typically lies in the range between 30-75° C., preferably between 45-60° C. In a preferred embodiment the process is carried at a temperature from 25° C. to 40° C., such as from 28° C. to 35° C., such as from 30° C. to 34° C., preferably around 32° C.

As disclosed above in the background art section, the use of proteases during fermentation is known in the art, however, according to the present invention an increased ethanol yield may be obtained when saccharification and/or fermentation is performed in the presence of an endo-protease and exo-protease mixture. In particular the present inventors have found that, the exo-protease should make up at least 5% (w/w) of the protease mixture on a total protease enzyme protein basis.

In one embodiment the exo-protease makes up at least 10% (w/w) of the protease mixture on a total protease enzyme protein basis, such as at least 15%, at least 20%, at least 25%, at least 30%, at least 35%, at least 40%, at least 45%, at least 50%, at least 55%, at least 60%, at least 65%, at least 70%, particularly at least 75%, more particularly the exo-protease makes up from between 5 to 95% (w/w) on a total protease enzyme protein basis, particularly 10 to 80% (w/w), particularly 15 to 70% (w/w), more particularly 20 to 60% (w/w), and even more particularly 25 to 50% (w/w) of the protease mixture in the composition on a total protease enzyme protein basis.

In another embodiment the endo-protease and exo-protease is present in a ratio of 5:2 micro grams enzyme protein (EP)/g dry solids (DS), particularly 5:3, more particularly 5:4. The proteases used in a process of the invention are selected from endo-peptidases (endo-proteases) and exo-peptidases (exo-proteases). Among endo-peptidases, serine proteases (EC 3.4.21) and metallo-proteases (EC 3.4.24) are especially relevant.

The endo-protease may be selected from the group consisting of serine proteases belonging to family S53, S8, or from metallo proteases belonging to family M35.

In another particular embodiment the endo-protease is selected from metallo-proteases (see Handbook of Proteolytic Enzymes, A. J. Barrett, N. D. Rawlings, J. F. Woessner (eds), Academic Press (1998)); in particular, the proteases of the invention are selected from the group consisting of:
(a) proteases belonging to the EC 3.4.24 metalloendopeptidases;
(b) metalloproteases belonging to the M group of the above Handbook;
(c) metalloproteases belonging to family M35 (as defined at pp. 1492-1495 of the above Handbook).

In one particular embodiment the endo-protease is selected from the M35 family, more particularly M35 protease derived from Thermoascus aurantiacus, the mature polypeptide of which comprises amino acids 1-177 of SEQ ID NO: 1 or a polypeptide having at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% identity to the polypeptide of SEQ ID NO: 1.

The exo-protease is preferably selected from a protease belonging to family S10, S53, M14, M28.

The exo-protease is in another embodiment selected from S53 exo-protease is derived from a strain of Aspergillus, Trichoderma, Thermoascus, or Thermomyces, particularly Aspergillus oryzae, Aspergillus niger, Trichoderma reesei, Thermoascus thermophilus, or Thermomyces lanuginosus.

In one particular embodiment the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2, or the polypeptide of SEQ ID NO: 3.

In one particular embodiment the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4.

Before initiating the process a slurry of starch-containing material, such as granular starch, having 10-55 w/w % dry solids (DS), preferably 25-45 w/w % dry solids, more preferably 30-40 w/w % dry solids of starch-containing material may be prepared. The slurry may include water and/or process waters, such as stillage (backset), scrubber water, evaporator condensate or distillate, side-stripper water from distillation, or process water from other fermentation product plants.

In a particular embodiment, the process of the invention further comprises, prior to the conversion of a starch-containing material to sugars/dextrins the steps of:

(x) reducing the particle size of the starch-containing material; and

(y) forming a slurry comprising the starch-containing material and water.

In an embodiment, the starch-containing material is milled to reduce the particle size. In an embodiment the particle size is reduced to between 0.05-3.0 mm, preferably 0.1-0.5 mm, or so that at least 30%, preferably at least 50%, more preferably at least 70%, even more preferably at least 90% of the starch-containing material fits through a sieve with a 0.05-3.0 mm screen, preferably 0.1-0.5 mm screen.

After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material are converted into a soluble starch hydrolyzate.

In an embodiment, the particle size is smaller than a #7 screen, e.g., a #6 screen. A #7 screen is usually used in conventional prior art processes.

Alpha-Amylase Present and/or Added in Liquefaction

Alpha-amylases for use in liquefaction are preferably bacterial acid stable alpha-amylases. Particularly the alpha-amylase is from an Exiguobacterium sp. or a Bacillus sp. such as e.g., Bacillus stearothermophilus or Bacillus licheniformis.

In an embodiment the alpha-amylase is from the genus Bacillus, such as a strain of Bacillus stearothermophilus, in particular a variant of a Bacillus stearothermophilus alpha-amylase, such as the one shown in SEQ ID NO: 3 in WO 99/019467 or SEQ ID NO: 12 herein.

In an embodiment the Bacillus stearothermophilus alpha-amylase has a double deletion of two amino acids in the region from position 179 to 182, more particularly a double deletion at positions I181+G182, R179+G180, G180+I181, R179+I181, or G180+G182, preferably I181+G182, and optionally a N193F substitution, (using SEQ ID NO: 12 for numbering).

In an embodiment the Bacillus stearothermophilus alpha-amylase has a substitution at position S242, preferably S242Q substitution.

In an embodiment the Bacillus stearothermophilus alpha-amylase has a substitution at position E188, preferably E188P substitution.

In an embodiment the alpha-amylase is selected from the group of Bacillus stearothermophilus alpha-amylase variants with the following mutations:
I181*+G182*+N193F+E129V+K177L+R179E;
I181*+G182*+N193F+V59A+Q89R+E129V+K177L+R179E+H208Y+K220P+N224L+Q254S;
I181*+G182*+N193F+V59A Q89R+E129V+K177L+R179E+Q254S+M284V; and
I181*+G182*+N193F+E129V+K177L+R179E+K220P+N224L+S242Q+Q254S (using SEQ ID NO: 12 for numbering).

In an embodiment the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 12.

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 12 herein, or variants thereof, are truncated in the C-terminal preferably to have around 490 amino acids, such as from 482-493 amino acids. Preferably the Bacillus stearothermophilus variant alpha-amylase is truncated, preferably after position 484 of SEQ ID NO: 12, particularly after position 485, particularly after position 486, particularly after position 487, particularly after position 488, particularly after position 489, particularly after position 490, particularly after position 491, particularly after position 492, more particularly after position 493.

Glucoamylase Present and/or Added in Saccharification and/or Fermentation

The carbohydrate-source generating enzyme present during saccharification may in one embodiment be a glucoamylase. A glucoamylase is present and/or added in saccharification and/or fermentation, preferably simultaneous saccharification and fermentation (SSF), in a process of the invention (i.e., saccharification and fermentation of ungelatinized or gelatinized starch material).

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, preferably P. sanguineus, or a strain of Gloeophyllum, such as G. serpiarium, G. abietinum or G. trabeum, or a strain of the Nigrofomes.

In an embodiment the glucoamylase is derived from Talaromyces, such as a strain of Talaromyces emersonii, such as the one shown in SEQ ID NO: 8.

In an embodiment the glucoamylase is selected from the group consisting of:

(i) a glucoamylase comprising the polypeptide of SEQ ID NO: 8;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576, or SEQ ID NO: 9 herein.

In an embodiment the glucoamylase is selected from the group consisting of:

(i) a glucoamylase comprising the polypeptide of SEQ ID NO: 9;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 9.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803.

In an embodiment the glucoamylase is derived from Gloeophyllum serpiarium, such as the one shown in SEQ ID NO: 10.

In an embodiment the glucoamylase is selected from the group consisting of:

(i) a glucoamylase comprising the polypeptide of SEQ ID NO: 10;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 10.

In another embodiment the glucoamylase is derived from Gloeophyllum trabeum such as the one shown in SEQ ID NO: 11. In an embodiment the glucoamylase is selected from the group consisting of:

(i) a glucoamylase comprising the polypeptide of SEQ ID NO: 11;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 11.

In an embodiment the glucoamylase is derived from Trametes, such as a strain of Trametes cingulata, such as the one shown in SEQ ID NO: 7.

In one embodiment the glucoamylase is selected from the group consisting of:

(i) a glucoamylase comprising the polypeptide of SEQ ID NO: 7;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7.

In an embodiment the glucoamylase is derived from a strain of the genus Nigrofomes, in particular a strain of Nigrofomes sp. disclosed in WO 2012/064351.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS, especially 0.1-0.5 AGU/g DS.

Commercially available compositions comprising glucoamylase include AMG 200L; AMG 300 L; SANT™ SUPER, SANT® EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

Alpha-Amylase Present and/or Added in Saccharification and/or Fermentation

In an embodiment an alpha-amylase is present and/or added in saccharification and/or fermentation in the processes of the invention. In a preferred embodiment the alpha-amylase is of fungal or bacterial origin. In a preferred embodiment the alpha-amylase is a fungal acid stable alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

In one embodiment the alpha-amylase is derived from the genus Aspergillus, especially a strain of A. terreus, A. niger, A. oryzae, A. awamori, or Aspergillus kawachii, or of the genus Rhizomucor, preferably a strain the Rhizomucor pusillus, or the genus Meripilus, preferably a strain of Meripilus giganteus.

In a preferred embodiment the alpha-amylase present and/or added in saccharification and/or fermentation is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-binding domain, such as the one shown in SEQ ID NO: 6 herein, or a variant thereof.

In an embodiment the alpha-amylase present and/or added in saccharification and/or fermentation is selected from the group consisting of:

(i) an alpha-amylase comprising the polypeptide of SEQ ID NO: 6;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

In a preferred embodiment the alpha-amylase is a variant of the alpha-amylase shown in SEQ ID NO: 6 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 6 for numbering).

In an embodiment the alpha-amylase is derived from a Rhizomucor pusillus with an Aspergillus niger glucoamylase linker and starch-binding domain (SBD), preferably disclosed as SEQ ID NO: 6, preferably having one or more of the following substitutions: G128D, D143N, preferably G128D+D143N (using SEQ ID NO: 6 for numbering), and wherein the alpha-amylase variant present and/or added in saccharification and/or fermentation has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6 herein.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1:1, such as from 100:2 to 100:50, such as from 100:3 to 100:70.

In one embodiment the alpha-amylase is present in an amount of 0.001 to 10 AFAU/g DS, preferably 0.01 to 5 AFAU/g DS, especially 0.3 to 2 AFAU/g DS or 0.001 to 1 FAU-F/g DS, preferably 0.01 to 1 FAU-F/g DS.

In a further embodiment the alpha-amylase and glucoamylase is added in a ratio of between 0.1 and 100 AGU/FAU-F, preferably 2 and 50 AGU/FAU-F, especially between 10 and 40 AGU/FAU-F when saccharification and fermentation are carried out simultaneously.

### Fermentation

The fermentation conditions are determined based on, e.g., the kind of plant material, the available fermentable sugars, the fermenting organism(s) and/or the desired fermentation product. One skilled in the art can easily determine suitable fermentation conditions. The fermentation may be carried out at conventionally used conditions. Preferred fermentation processes are anaerobic processes.

For example, fermentations may be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms” section above.

For ethanol production using yeast, the fermentation may go on for 24 to 96 hours, in particular for 35 to 60 hours. In an embodiment the fermentation is carried out at a temperature between 20 to 40° C., preferably 26 to 34° C., in particular around 32° C.

The fermentation may include, in addition to a fermenting microorganisms (e.g., yeast), nutrients, and additional enzymes, including phytases. The use of yeast in fermentation is well known in the art.

Other fermentation products may be fermented at temperatures known to the skilled person in the art to be suitable for the fermenting organism in question.

Fermentation is typically carried out at a pH in the range between 3 and 7, preferably from pH 3.5 to 6, more preferably pH 4 to 5. Fermentations are typically ongoing for 6-96 hours.

The processes of the invention may be performed as a batch or as a continuous process. Fermentations may be conducted in an ultrafiltration system wherein the retentate is held under recirculation in the presence of solids, water, and the fermenting organism, and wherein the permeate is the desired fermentation product containing liquid. Equally contemplated are methods/processes conducted in continuous membrane reactors with ultrafiltration membranes and where the retentate is held under recirculation in presence of solids, water, and the fermenting organism(s) and where the permeate is the fermentation product containing liquid.

After fermentation the fermenting organism may be separated from the fermented slurry and recycled.

### Starch-Containing Materials

Any suitable starch-containing starting material may be used in a process of the present invention. In one embodiment the starch-containing material is granular starch. In another embodiment the starch-containing material is derived from whole grain. The starting material is generally selected based on the desired fermentation product. Examples of starch-containing starting materials, suitable for use in the processes of the present invention, include barley, beans, cassava, cereals, corn, milo, peas, potatoes, rice, rye, sago, sorghum, sweet potatoes, tapioca, wheat, and whole grains, or any mixture thereof. The starch-containing material may also be a waxy or non-waxy type of corn and barley. In a preferred embodiment the starch-containing material is corn. In a preferred embodiment the starch-containing material is wheat.

### Fermentation Products

The term “fermentation product” means a product produced by a method or process including fermenting using a fermenting organism. Fermentation products include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. In an preferred embodiment the fermentation product is ethanol.

### Fermenting Organisms

The term “fermenting organism” refers to any organism, including bacterial and fungal organisms, such as yeast and filamentous fungi, suitable for producing a desired fermentation product. Suitable fermenting organisms are able to ferment, i.e., convert, fermentable sugars, such as arabinose, fructose, glucose, maltose, mannose, or xylose, directly or indirectly into the desired fermentation product.

Examples of fermenting organisms include fungal organisms such as yeast. Preferred yeast include strains of Saccharomyces, in particular Saccharomyces cerevisiae or Saccharomyces uvarum; strains of Pichia, in particular Pichia stipitis such as Pichia stipitis CBS 5773 or Pichia pastoris; strains of Candida, in particular Candida arabinofermentans, Candida boidinii, Candida diddensii, Candida shehatae, Candida sonorensis, Candida tropicalis, or Candida utilis. Other fermenting organisms include strains of Hansenula, in particular Hansenula anomala or Hansenula polymorpha; strains of Kluyveromyces, in particular Kluyveromyces fragilis or Kluyveromyces marxianus; and strains of Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes, and strains of Thermoanaerobacter, in particular Thermoanaerobacter BG1L1 (Appl. Microbiol. Biotech. 77: 61-86), Thermoanarobacter ethanolicus, Thermoanaerobacter mathranii, or Thermoanaerobacter thermosaccharolyticum. Strains of Lactobacillus are also envisioned as are strains of Corynebacterium glutamicum R, Bacillus thermoglucosidaisus, and Geobacillus thermoglucosidasius.

In an embodiment, the fermenting organism is a C6 sugar fermenting organism, such as a strain of, e.g., Saccharomyces cerevisiae.

In an embodiment, the fermenting organism is a C5 sugar fermenting organism, such as a strain of, e.g., Saccharomyces cerevisiae.

The amount of starter yeast employed in fermentation is an amount effective to produce a commercially significant amount of ethanol in a suitable amount of time, (e.g., to produce at least 10% ethanol from a substrate having between 25-40% DS in less than 72 hours). Yeast cells are generally supplied in amounts of about 10⁴ to about 10¹², and preferably from about 10⁷ to about 10¹⁰, especially about 5×10⁷ viable yeast count per mL of fermentation broth. After yeast is added to the mash, it is typically subjected to fermentation for about 24-96 hours, e.g., 35-60 hours. The temperature is between about 26-34° C., typically at about 32° C., and the pH is from pH 3-6, e.g., around pH 4-5.

Yeast is the preferred fermenting organism for ethanol fermentation. Preferred are strains of Saccharomyces, especially strains of the species Saccharomyces cerevisiae, preferably strains which are resistant towards high levels of ethanol, i.e., up to, e.g., about 10, 12, 15 or 20 vol. % or more ethanol.

In an embodiment, the C5 utilizing yeast is a Saccharomyces cerevisiae strain disclosed in WO 2004/085627.

In an embodiment, the fermenting organism is a C5 eukaryotic microbial cell concerned in WO 2010/074577 (Nedalco).

In an embodiment, the fermenting organism is a transformed C5 eukaryotic cell capable of directly isomerize xylose to xylulose disclosed in US 2008/0014620.

In an embodiment, the fermenting organism is a C5 sugar fermentating cell disclosed in WO 2009/109633.

Commercially available yeast include LNF SA-1, LNF BG-1, LNF PE-2, and LNF CAT-1 (available from LNF Brazil), RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

The fermenting organism capable of producing a desired fermentation product from fermentable sugars is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

### Recovery

Subsequent to fermentation, the fermentation product may be separated from the fermentation medium. Thus in one embodiment the fermentation product is recovered after fermentation. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

### Enzyme Compositions

The present invention also relates to a composition comprising a mixture of endo-protease and exo-protease, and wherein the exo-protease makes up at least 5% (w/w) of the protease in the mixture on a total protease enzyme protein basis, such as at least 10%, at least 15%, at least 20%, at least 25%, at least 30%, at least 35%, at least 40%, at least 45%, at least 50%, at least 55%, at least 60%, at least 65%, at least 70%, particularly at least 75%, more particularly the exo-protease makes up from between 5 to 95% (w/w) of the protease in the mixture on a total protease enzyme protein basis, particularly 10 to 80% (w/w), particularly 15 to 70% (w/w), more particularly 20 to 60% (w/w), and even more particularly 25 to 50% (w/w) of the protease mixture in the composition on a total protease enzyme protein basis.

In one embodiment the endo-protease is derived from proteases belonging to family S53, S8, M35, or A1 and the exo-protease is derived from proteases belonging to family S10, S53, M14, or M28.

The endo-protease is preferable selected from from the M35 family, more particularly M35 protease derived from Thermoascus aurantiacus.

In a particular embodiment the M35 metallo-protease is derived from Thermoascus aurantiacus, such as e.g., the mature polypeptide which comprises amino acids 1-177 of SEQ ID NO: 1 or a polypeptide having at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% identity to the polypeptide of SEQ ID NO: 1.

The exo-protease is preferably selected from a protease belonging to family S10, S53, M14, M28, particularly S53 exo-protease from Aspergillus, Trichoderma, Thermoascus, or Thermomyces, particularly Aspergillus oryzae, Trichoderma reesei, Thermoascus thermophilus, or Thermomyces lanuginosus.

In one specific embodiment the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2, or the polypeptide of SEQ ID NO: 3.

In another specific embodiment the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4.

In one particular embodiment the endo-protease is a S53 protease from Thermoascus aurantiacus, such as the one disclosed in SEQ ID NO: 1, and the exo-protease is a S53 protease from Aspergillus, Trichoderma, Thermoascus, or Thermomyces, particularly Aspergillus niger, Trichoderma reesei, selected from the group consisting of SEQ ID NO: 3, and 4.

The compositions may comprise the proteases as the major enzymatic components. Alternatively, the compositions may comprise multiple enzymatic activities, such as the end-protease/exo-protease and one or more (e.g., several) enzymes selected from the group consisting of hydrolase, isomerase, ligase, lyase, oxidoreductase, or transferase, e.g., an alpha-galactosidase, alpha-glucosidase, aminopeptidase, alpha-amylase, beta-amylase, pullulanase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, protease, ribonuclease, transglutaminase, or xylanase. In one embodiment the composition further comprises a carbohydrate-source generating enzyme and optionally an alpha-amylase. In one particular embodiment the carbohydrate-source generating enzyme is selected from the group consisting of glucoamylase, alpha-glucosidase, maltogenic amylase, pullulanase and beta-amylase.

In particular, the carbohydrase-source generating enzyme is a glucoamylase and is present in an amount of 0.001 to 10 AGU/g DS, preferably from 0.01 to 5 AGU/g DS, especially 0.1 to 0.5 AGU/g DS.

In an embodiment the glucoamylase comprised in the composition is of fungal origin, preferably derived from a strain of Aspergillus, preferably Aspergillus niger, Aspergillus oryzae, or Aspergillus awamori, a strain of Trichoderma, especially T. reesei, a strain of Talaromyces, especially Talaromyces emersonii; or a strain of Athelia, especially Athelia rolfsii; a strain of Trametes, preferably Trametes cingulata; a strain of the genus Gloeophyllum, e.g., a strain of Gloeophyllum sepiarum or Gloeophyllum trabeum; a strain of the genus Pycnoporus, e.g., a strain of Pycnoporus sanguineus; or a strain of the Nigrofomes, or a mixture thereof.

In an embodiment the glucoamylase is derived from Trametes, such as a strain of Trametes cingulata, such as the one shown in SEQ ID NO: 7.

In an embodiment the glucoamylase is selected from the group consisting of:

(i) a glucoamylase comprising the polypeptide of SEQ ID NO: 7;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7.

In an embodiment the glucoamylase is derived from Talaromyces, such as a strain of Talaromyces emersonii, such as the one shown in SEQ ID NO: 8,

In an embodiment the glucoamylase is selected from the group consisting of:

(i) a glucoamylase comprising the polypeptide of SEQ ID NO: 8;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576.

In an embodiment the glucoamylase is selected from the group consisting of:

(i) a glucoamylase comprising the polypeptide of SEQ ID NO: 9;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 9.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803.

In an embodiment the glucoamylase is derived from Gloeophyllum serpiarium, such as the one shown in SEQ ID NO: 10.

In an embodiment the glucoamylase is selected from the group consisting of:

(i) a glucoamylase comprising the polypeptide of SEQ ID NO: 10;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 10.

In another embodiment the glucoamylase is derived from Gloeophyllum trabeum such as the one shown in SEQ ID NO: 11.

In an embodiment the glucoamylase is selected from the group consisting of:

(i) a glucoamylase comprising the polypeptide of SEQ ID NO: 11;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 11.

In an embodiment the glucoamylase is derived from a strain of the genus Nigrofomes, in particular a strain of Nigrofomes sp. disclosed in WO 2012/064351.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SANT™ SUPER, SANT™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont); AMIGASE™ and AMIGASE™ PLUS (from DSM); G-ZYME™ G900, G-ZYME™ and G990 ZR (from DuPont).

In addition to a glucoamylase the composition may further comprise an alpha-amylase. Particularly the alpha-amylase is an acid fungal alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

Preferably the acid fungal alpha-amylase is derived from the genus Aspergillus, especially a strain of A. terreus, A. niger, A. oryzae, A. awamori, or Aspergillus kawachii, or from the genus Rhizomucor, preferably a strain the Rhizomucor pusillus, or the genus Meripilus, preferably a strain of Meripilus giganteus.

In a preferred embodiment the alpha-amylase is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-binding domain, such as the one shown in SEQ ID NO: 6 herein, or a variant thereof.

In an embodiment the alpha-amylase is selected from the group consisting of:

(i) an alpha-amylase comprising the polypeptide of SEQ ID NO: 6;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

In a preferred embodiment the alpha-amylase is a variant of the alpha-amylase shown in SEQ ID NO: 9 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 6 for numbering).

In an embodiment the alpha-amylase is derived from a Rhizomucor pusillus with an Aspergillus niger glucoamylase linker and starch-binding domain (SBD), preferably disclosed as SEQ ID NO: 6, preferably having one or more of the following substitutions: G128D, D143N, preferably G128D+D143N (using SEQ ID NO: 6 for numbering), and wherein the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1:1, such as from 100:2 to 100:50, such as from 100:3 to 100:70.

The compositions may be prepared in accordance with methods known in the art and may be in the form of a liquid or a dry composition. For instance, the composition may be in the form of granulate or microgranulate. The variant may be stabilized in accordance with methods known in the art.

The compositions may be prepared in accordance with methods known in the art and may be in the form of a liquid or a dry composition. The compositions may be stabilized in accordance with methods known in the art.

The enzyme composition of the present invention may be in any form suitable for use, such as, for example, a crude fermentation broth with or without cells removed, a cell lysate with or without cellular debris, a semi-purified or purified enzyme composition, or a host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

### Uses of the Composition According to the Invention

The compositions according to the invention are contemplated for use in saccharification of starch. In one aspect the present invention thus relates to a use of the composition according to the present invention in saccharification of a starch containing material.

In one embodiment the use further comprises fermenting the saccharified starch containing material to produce a fermentation product. The starch material may be gelatinized or ungelatinized starch. Particularly the fermentation product is alcohol, more particularly ethanol.

In a particular embodiment saccharification and fermentation is performed simultaneously.

The invention is further disclosed in the below list of preferred embodiments.

### Embodiment 1

A process for producing a fermentation product from starch-containing material comprising:

a) saccharifying the starch-containing material at a temperature below the initial gelatinization temperature of said starch-containing material using a carbohydrate-source generating enzymes; and
b) fermenting using a fermenting organism; wherein
steps a) and/or b) is performed in the presence of an endo-protease and an exo-protease mixture, and wherein the exo-protease makes up at least 5% (w/w) of the protease mixture on a total protease enzyme protein basis.

### Embodiment 2

A process for producing a fermentation product from starch-containing material comprising the steps of:

(a) liquefying starch-containing material at a temperature above the initial gelatinization temperature of said starch-containing material in the presence of an alpha-amylase;

(b) saccharifying the liquefied material obtained in step (a) using a carbohydrate-source generating enzyme;

(c) fermenting using a fermenting organism;

wherein steps b) and/or c) is performed in the presence of an endo-protease and an exo-protease mixture, and wherein the exo-protease makes up at least 5% (w/w) of the protease mixture on a total protease enzyme protein basis.

### Embodiment 3

The process according to embodiments 1 or 2, wherein saccharification and fermentation is performed simultaneously.

### Embodiment 4

The process according to any of the preceding embodiments, wherein the exo-protease makes up at least 10% (w/w) of the protease mixture on a total protease enzyme protein basis, such as at least 15%, at least 20%, at least 25%, at least 30%, at least 35%, at least 40%, at least 45%, at least 50%, at least 55%, at least 60%, at least 65%, at least 70%, particularly at least 75%, more particularly the exo-protease makes up from between 5 to 95% (w/w) on a total protease enzyme protein basis, particularly 10 to 80% (w/w), particularly 15 to 70% (w/w), more particularly 20 to 60% (w/w), and even more particularly 25 to 50% (w/w) of the protease mixture in the composition on a total protease enzyme protein basis.

### Embodiment 5

The process according to any of the preceding embodiments, wherein the endo-protease and exo-protease is present in a ratio of 5:2 micro grams enzyme protein (EP)/g dry solids (DS), particularly 5:3, more particularly 5:4.

### Embodiment 6

The process according to any of embodiments 1-5, wherein the endo-protease is derived from proteases belonging to family S53, S8, M35, A1.

### Embodiment 7

The process according to any of embodiments 1-5, wherein the exo-protease is derived from proteases belonging to family S10, S53, M14, M28.

### Embodiment 8

The process of embodiment 7, wherein the endo-protease is selected from the M35 family, more particularly M35 protease derived from Thermoascus aurantiacus, the mature polypeptide of which comprises amino acids 1-177 of SEQ ID NO: 1 or a polypeptide having at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% identity to the polypeptide of SEQ ID NO: 1.

### Embodiment 9

The process according to embodiment 8, wherein the S53 exo-protease is derived from a strain of Aspergillus, Trichoderma, Thermoascus, or Thermomyces, particularly Aspergillus oryzae, Aspergillus niger, Trichoderma reesei, Thermoascus thermophilus, or Thermomyces lanuginosus.

### Embodiment 10

The process according to embodiment 9, wherein the S53 protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2, or the polypeptide of SEQ ID NO: 3.

### Embodiment 11

The process according to embodiments 9, wherein the S53 protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4.

### Embodiment 12

The process of any of the preceding embodiments, wherein an alpha-amylase is present or added during saccharification and/or fermentation.

### Embodiment 13

The process according to embodiment 12, wherein the alpha-amylase is an acid alpha-amylase, preferably an acid fungal alpha-amylase.

### Embodiment 14

The process according to embodiment 13, wherein the alpha-amylase is derived from the genus Aspergillus, especially a strain of A. terreus, A. niger, A. oryzae, A. awamori, or Aspergillus kawachii, or of the genus Rhizomucor, preferably a strain the Rhizomucor pusillus, or the genus Meripilus, preferably a strain of Meripilus giganteus.

### Embodiment 15

The process according to embodiment 14, wherein the alpha-amylase present in saccharification and/or fermentation is derived from a strain of the genus Rhizomucor, preferably a strain of Rhizomucor pusillus, such as a Rhizomucor pusillus alpha-amylase hybrid having a linker and starch-binding domain from an Aspergillus niger glucoamylase.

### Embodiment 16

The process of embodiment 15, wherein the alpha-amylase present in saccharification and/or fermentation is selected from the group consisting of:

(i) an alpha-amylase comprising the polypeptide of SEQ ID NO: 6;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

### Embodiment 17

The process of embodiment 16, wherein the alpha-amylase is derived from a Rhizomucor pusillus with an Aspergillus niger glucoamylase linker and starch-binding domain (SBD), preferably disclosed as SEQ ID NO: 6, preferably having one or more of the following substitutions: G128D, D143N, preferably G128D+D143N.

### Embodiment 18

The process of any of embodiments 12-17, wherein the alpha-amylase is present in an amount of 0.001 to 10 AFAU/g DS, preferably 0.01 to 5 AFAU/g DS, especially 0.3 to 2 AFAU/g DS or 0.001 to 1 FAU-F/g DS, preferably 0.01 to 1 FAU-F/g DS.

### Embodiment 19

The process of any of embodiments 1-18, wherein the carbohydrate-source generating enzyme is selected from the group consisting of glucoamylase, alpha-glucosidase, maltogenic amylase, pullulanase, and beta-amylase.

### Embodiment 20

The process of any of embodiments 1-19, wherein the carbohydrase-source generating enzyme is a glucoamylase and is present in an amount of 0.001 to 10 AGU/g DS, preferably from 0.01 to 5 AGU/g DS, especially 0.1 to 0.5 AGU/g DS.

### Embodiment 21

The process of any of embodiments 18-20, wherein the alpha-amylase and glucoamylase is added in a ratio of between 0.1 and 100 AGU/FAU-F, preferably 2 and 50 AGU/FAU-F, especially between 10 and 40 AGU/FAU-F when saccharification and fermentation are carried out simultaneously.

### Embodiment 22

The process of any of embodiments 19-21, wherein the glucoamylase is derived from a strain of Aspergillus, preferably Aspergillus niger or Aspergillus awamori, a strain of Talaromyces, especially Talaromyces emersonii; or a strain of Athelia, especially Athelia rolfsii; a strain of Trametes, preferably Trametes cingulata; a strain of the genus Gloeophyllum, e.g., a strain of Gloeophyllum sepiarium or Gloeophyllum trabeum; a strain of the genus Pycnoporus, e.g., a strain of Pycnoporus sanguineus; or a mixture thereof.

### Embodiment 23

The process of embodiment 22, wherein the glucoamylase is derived from Trametes, such as a strain of Trametes cingulata, such as the one shown in SEQ ID NO: 7.

### Embodiment 24

The process of embodiment 23, wherein the glucoamylase is selected from the group consisting of:

(i) a glucoamylase comprising the polypeptide of SEQ ID NO: 7;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7.

### Embodiment 25

The process of embodiment 22, wherein the glucoamylase is derived from Talaromyces, such as a strain of Talaromyces emersonii, such as the one shown in SEQ ID NO: 8.

### Embodiment 26

The process of embodiment 25, wherein the glucoamylase is selected from the group consisting of:

(i) a glucoamylase comprising the polypeptide of SEQ ID NO: 8;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8.

### Embodiment 27

The process of embodiment 22, wherein the glucoamylase is derived from a strain of the genus Pycnoporus, such as a strain of Pycnoporus sanguineus such as the one shown in SEQ ID NO: 9.

### Embodiment 28

The process of embodiment 27, wherein the glucoamylase is selected from the group consisting of:

(i) a glucoamylase comprising the polypeptide of SEQ ID NO: 9;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 9.

### Embodiment 29

The process of embodiment 22, wherein the glucoamylase is derived from a strain of the genus Gloeophyllum, such as a strain of Gloeophyllum sepiarium shown in SEQ ID NO: 10.

### Embodiment 30

The process of embodiment 29, wherein the glucoamylase is selected from the group consisting of:

(i) a glucoamylase comprising the polypeptide of SEQ ID NO: 10;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 10.

### Embodiment 31

The process of embodiment 22, wherein the glucoamylase is derived from a strain of the genus Gloeophyllum, such as a strain of Gloeophyllum trabeum such as the one shown in SEQ ID NO: 11.

### Embodiment 32

The process of embodiment 22, wherein the glucoamylase is selected from the group consisting of:

(i) a glucoamylase comprising the polypeptide of SEQ ID NO: 11;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 11.

### Embodiment 33

The process of any of embodiments 1-32, wherein the fermentation product is recovered after fermentation.

### Embodiment 34

The process of any of embodiments 1-33, wherein the fermentation product is an alcohol, preferably ethanol, especially fuel ethanol, potable ethanol and/or industrial ethanol.

### Embodiment 35

The process of any of embodiments 1-34, wherein the fermenting organism is yeast, preferably a strain of Saccharomyces, especially a strain of Saccharomyces cerevisiae.

### Embodiment 36

The process of embodiment 1, wherein the starch-containing material is granular starch.

### Embodiment 37

The process of embodiment 36, wherein the starch-containing material is derived from whole grain.

### Embodiment 38

The process of any of embodiments 1-37, wherein the starch-containing material is derived from corn, wheat, barley, rye, milo, sago, cassava, tapioca, sorghum, rice or potatoes.

### Embodiment 39

The process of any of embodiments 1-38, wherein fermentation is carried out at a pH in the range between 3 and 7, preferably from 3.5 to 6, or more preferably from 4 to 5.

### Embodiment 40

The process of any of embodiments 1-39, wherein the process is carried out for between 1 to 96 hours, preferably is from 6 to 72 hours.

### Embodiment 41

The process of any of embodiments 1-40, wherein the dry solid content of the starch-containing material is in the range from 10-55 w/w-%, preferably 25-45 w/w-%, more preferably 30-40 w/w-%.

### Embodiment 42

The process of any of embodiments 1-41, wherein the starch-containing material is prepared by reducing the particle size of starch-containing material to a particle size of 0.1-0.5 mm.

### Embodiment 43

The process of embodiment 3, wherein the temperature during simultaneous saccharification and fermentation is between 25° C. and 40° C., such as between 28° C. and 35° C., such as between 30° C. and 34° C., such as around 32° C.

### Embodiment 44

The process of embodiment 3, wherein the pH during simultaneous saccharification and fermentation is selected from the range 3-7, preferably 4.0-6.5, more particularly 4.5-5.5, such as pH 5.0.

### Embodiment 45

The process of any of embodiments 2-44, wherein liquefaction is carried out at pH 4.0-6.5, preferably at a pH from 4.5 to 5.5, such as pH 5.0.

### Embodiment 46

The process of any of embodiments 2-45, wherein the temperature in liquefaction is in the range from 70−95° C., preferably 80-90° C., such as around 85° C.

### Embodiment 47

The process of embodiments 1 or 2, further comprising, prior to the step (a), the steps of:

x) reducing the particle size of starch-containing material;

y) forming a slurry comprising the starch-containing material and water.

### Embodiment 48

The process of any of embodiments 1-47, wherein a pullulanase is present i) during fermentation, and/or ii) before, during, and/or after liquefaction.

### Embodiment 49

A composition comprising a mixture of endo-protease and exo-protease, and wherein the exo-protease makes up at least 5% (w/w) of the protease in the mixture on a total protease enzyme protein basis, such as at least 10%, at least 15%, at least 20%, at least 25%, at least 30%, at least 35%, at least 40%, at least 45%, at least 50%, at least 55%, at least 60%, at least 65%, at least 70%, particularly at least 75%, more particularly the exo-protease makes up from between 5 to 95% (w/w) of the protease in the mixture on a total protease enzyme protein basis, particularly 10 to 80% (w/w), particularly 15 to 70% (w/w), more particularly 20 to 60% (w/w), and even more particularly 25 to 50% (w/w) of the protease mixture in the composition on a total protease enzyme protein basis.

### Embodiment 50

The composition of embodiment 49, wherein the endo-protease is derived from proteases belonging to family S53, S8, M35, or A1 and the exo-protease is derived from proteases belonging to family S10, S53, M14, or M28.

### Embodiment 51

The composition of embodiments 50, wherein the S53 endo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 1.

### Embodiment 52

The composition according to embodiment 50, wherein wherein the S53 exo-protease is derived from a strain of Aspergillus, Trichoderma, Thermoascus, or Thermomyces, particularly Aspergillus oryzae, Aspergillus niger, Trichoderma reesei, Thermoascus thermophilus, or Thermomyces lanuginosus.

### Embodiment 53

The composition according to embodiments 52, wherein the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2, or the polypeptide of SEQ ID NO: 3.

### Embodiment 54

The composition according to embodiments 52, wherein the S53 exo-protease is a polypeptide having serine protease activity, selected from a polypeptide having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4.

### Embodiment 55

The composition of any of the embodiments 49-54, further comprising a carbohydrate-source generating enzyme selected from the group of glucoamylase, alpha-glucosidase, maltogenic amylase, and beta-amylase.

### Embodiment 56

The composition of embodiment 55, wherein the carbohydrate-source generating enzyme is selected from the group of glucoamylases derived from a strain of Aspergillus, preferably Aspergillus niger or Aspergillus awamori, a strain of Trichoderma, especially T. reesei, a strain of Talaromyces, especially Talaromyces emersonii; or a strain of Athelia, especially Athelia rolfsii; a strain of Trametes, preferably Trametes cingulata; a strain of the genus Gloeophyllum, e.g., a strain of Gloeophyllum sepiarum or Gloeophyllum trabeum; a strain of the genus Pycnoporus, e.g., a strain of Pycnoporus sanguineus; or a mixture thereof.

### Embodiment 57

The composition of any of embodiments 49-56, further comprising an alpha-amylase selected from the group of fungal alpha-amylases, preferably derived from the genus Aspergillus, especially a strain of Aspergillus terreus, Aspergillus niger, Aspergillus oryzae, Aspergillus awamori, or Aspergillus kawachii, or of the genus Rhizomucor, preferably a strain the Rhizomucor pusillus, or the genus Meripilus, preferably a strain of Meripilus giganteus.

### Embodiment 58

A use of the composition according to any of embodiments 49-57 in saccharification of a starch containing material.

### Embodiment 59

The use according to embodiment 58, further comprising fermenting the saccharified starch containing material to produce a fermentation product.

### Embodiment 60

The use according to any of the embodiments 58-59, wherein the starch material is gelatinized or ungelatinized starch.

### Embodiment 61

The use according to any of the embodiments 58-60, wherein the fermentation product is alcohol, particularly ethanol.

### Embodiment 62

The use according to any of embodiments 58-61, wherein saccharification and fermentation is performed simultaneously.

The present invention is further described by the following examples that should not be construed as limiting the scope of the invention.

### EXAMPLES

### Enzyme Assays

### Protease Assays

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

### Kinetic Suc-AAPF-pNA Assay:

pNA substrate: Suc-AAPF-pNA (Bachem L−1400).
Temperature: Room temperature (25° C.)
Assay buffers: 100 mM succinic acid, 100 mM HEPES, 100 mM CHES, 100 mM CABS, 1 mM CaCl₂, 150 mM KCl, 0.01% Triton X-100 adjusted to pH-values 2.0, 3.0, 4.0, 5.0, 6.0, 7.0, 8.0, 9.0, 10.0, and 11.0 with HCl or NaOH.
   20 μl protease sample (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD₄₀₅ was monitored as a measure of the protease activity.

### Endpoint Suc-AAPF-pNA Assay:

pNA substrate: Suc-AAPF-pNA (Bachem L-1400).
Temperature: controlled (assay temperature).
Assay buffer: 100 mM succinic acid, 100 mM HEPES, 100 mM CHES, 100 mM CABS, 1 mM CaCl₂, 150 mM KCl, 0.01% Triton X-100, pH 4.0

200 μl pNA substrate (50 mg dissolved in 1.0 ml DMSO and further diluted 45× with the Assay buffer) were pipetted in an Eppendorf tube and placed on ice. 20 μl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate (1400 rpm). The incubation was stopped by transferring the tube back to the ice bath and adding 600 μl 500 mM H₃BO₃/NaOH, pH 9.7. The tube was mixed and 200 μl mixture was transferred to a microtiter plate, which was read at OD₄₀₅. A buffer blind was included in the assay (instead of enzyme). OD₄₀₅(Sample)−OD₄₀₅(Blind) was a measure of protease activity.

Protazyme AK assay:
Substrate: Protazyme AK tablet (cross-linked and dyed casein; from Megazyme)
Temperature: controlled (assay temperature).
Assay buffer: 100 mM succinic acid, 100 mM HEPES, 100 mM CHES, 100 mM CABS, 1 mM CaCl₂, 150 mM KCl, 0.01% Triton X-100, pH 6.5.

A Protazyme AK tablet was suspended in 2.0 ml 0.01% Triton X-100 by gentle stirring. 500 μl of this suspension and 500 μl assay buffer were dispensed in an Eppendorf tube and placed on ice. 20 μl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate (1400 rpm). The incubation was stopped by transferring the tube back to the ice bath. Then the tube was centrifuged in an ice cold centrifuge for a few minutes and 200 μl supernatant was transferred to a microtiter plate, which was read at OD₆₅₀. A buffer blind was included in the assay (instead of enzyme). OD₆₅₀(Sample)−OD₆₅₀(Blind) was a measure of protease activity.

### Kinetic Suc-AAPX-pNA Assay:

pNA substrates: Suc-AAPA-pNA (Bachem L-1775)
   Suc-AAPR-pNA (Bachem L-1720)
   Suc-AAPD-pNA (Bachem L-1835)
   Suc-AAPI-pNA (Bachem L-1790)
   Suc-AAPM-pNA (Bachem L-1395)
   Suc-AAPV-pNA (Bachem L-1770)
   Suc-AAPL-pNA (Bachem L-1390)
   Suc-AAPE-pNA (Bachem L-1710)
   Suc-AAPK-pNA (Bachem L-1725)
   Suc-AAPF-pNA (Bachem L-1400)
Temperature: Room temperature (25° C.)
Assay buffer: 100 mM succinic acid, 100 mM HEPES, 100 mM CHES, 100 mM CABS, 1 mM CaCl₂, 150 mM KCl, 0.01% Triton X-100, pH 4.0 or pH 9.0.
   20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD₄₀₅ was monitored as a measure of the protease activity.
   o-Phthaldialdehyde (OPA) Assay:
   This assay detects primary amines and hence cleavage of peptide bonds by a protease can be measured as the difference in absorbance between a protease treated sample and a control sample. The assay is conducted essentially according to Nielsen et al. (Nielsen, P M, Petersen, D, Dampmann, C. Improved method for determining food protein degree of hydrolysis. J Food Sci, 2001, 66: 642-646).
   500 μl of sample is filtered through a 100 kDa Microcon centrifugal filter (60 min, 11,000 rpm, 5° C.). The samples are diluted appropriately (e.g. 10, 50 or 100 times) in deionizer water and 25 μl of each sample is loaded into a 96 well microtiter plate (5 replicates). 200 μl OPA reagent (100 mM di-sodium tetraborate decahydrate, 3.5 mM sodium dodecyl sulphate (SDS), 5.7 mM di-thiothreitol (DDT), 6 mM o-phthaldialdehyde) is dispensed into all wells, the plate is shaken (10 sec, 750 rpm) and absorbance measured at 340 nm.

### Assays for Glucoamylase Activity

### Glucoamylase Units, AGU

The Glucoamylase Unit (AGU) is defined as the amount of enzyme, which hydrolyses 1 micromole maltose per minute under the standard conditions (37° C., pH 4.3, substrate: maltose 100 mM, buffer: acetate 0.1 M, reaction time 6 minutes as set out in the glucoamylase incubation below), thereby generating glucose.

| glucoamylase incubation: | | |
|---|---|---|
| Substrate: | maltose 100 | mM |
| Buffer: | acetate 0.1M | |
| pH: | 4.30 ± 0.05 | |
| Incubation temperature: | 37° C. ± 1 | |
| Reaction time: | 6 | minutes |
| Enzyme working range: | 0.5-4.0 | AGU/mL |

The analysis principle is described by 3 reaction steps:
Step 1 is an enzyme reaction:

Glucoamylase (AMG), EC 3.2.1.3 (exo-alpha-1,4-glucan-glucohydrolase), hydrolyzes maltose to form alpha-D-glucose. After incubation, the reaction is stopped with NaOH.

Steps 2 and 3 result in an endpoint reaction:

Glucose is phosphorylated by ATP, in a reaction catalyzed by hexokinase. The glucose-6-phosphate formed is oxidized to 6-phosphogluconate by glucose-6-phosphate dehydrogenase. In this same reaction, an equimolar amount of NAD+ is reduced to NADH with a resulting increase in absorbance at 340 nm. An autoanalyzer system such as Konelab 30 Analyzer (Thermo Fisher Scientific) may be used.

Color reaction

| Tris | approx. 35 | mM |
|---|---|---|
| ATP | 0.7 | mM |
| NAD⁺ | 0.7 | mM |
| Mg²⁺ | 1.8 | mM |
| Hexokinase | >850 | U/L |
| Glucose-6-P-DH | >850 | U/L |
| pH | approx. 7.8 | |
| Temperature | 37.0° C. ± 1.0° C. | |
| Reaction time | 420 | sec |
| Wavelength | 340 | nm |

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

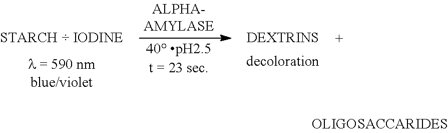

### Standard Conditions/Reaction Conditions

Substrate: Soluble starch, approx. 0.17 g/L
Buffer: Citrate, approx. 0.03 M
Iodine (I2): 0.03 g/L
CaCl₂: 1.85 mM
pH: 2.50±0.05
Incubation 40° C. temperature:
Reaction time: 23 seconds
Wavelength: 590 nm
Enzyme 0.025 AFAU/mL concentration:
Enzyme working 0.01-0.04 AFAU/mL range:
   A folder EB-SM-0259.02/01 describing this analytical method in more detail is available upon request to Novozymes A/S, Denmark, which folder is hereby included by reference.

### Determination of FAU-F

FAU-F Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Alpha-Amylase Activity (KNU-A)

Alpha amylase activity is measured in KNU(A) Kilo Novozymes Units (A), relative to an enzyme standard of a declared strength.

Alpha amylase in samples and α-glucosidase in the reagent kit hydrolyze the substrate (4,6-ethylidene(G₇)-p-nitrophenyl(G₁)-α,D-maltoheptaoside (ethylidene-G₇PNP) to glucose and the yellow-colored p-nitrophenol.

The rate of formation of p-nitrophenol can be observed by Konelab 30. This is an expression of the reaction rate and thereby the enzyme activity.

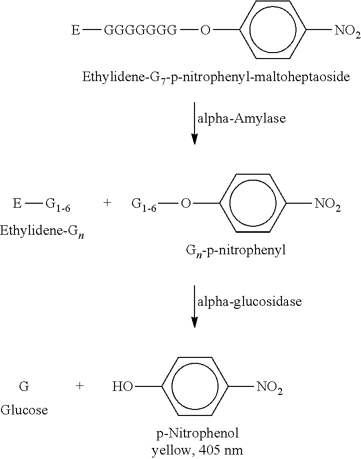

The enzyme is an alpha-amylase with the enzyme classification number EC 3.2.1.1.

| Parameter | Reaction conditions | |
|---|---|---|
| Temperature | 37° | C. |
| pH | 7.00 (at 37° C.) | |
| Substrate conc. | Ethylidene-G₇PNP, R2: 1.86 mM | |
| Enzyme conc. | 1.35-4.07 | KNU(A)/L |
| (conc. of high/low standard in | | |
| reaction mixture) | | |
| Reaction time | 2 | min |
| Interval kinetic measuring | 7/18 | sec. |
| time | | |
| Wave length | 405 | nm |

| | |
|---|---|
| Conc. of reagents/chemicals | α-glucosidase, R1: ≥3.39 kU/L |
| critical for the analysis | |

### Enzymes

Alpha-Amylase 369 (AA369): Bacillus stearothermophilus alpha-amylase with the mutations: I181*+G182*+N193F+V59A+Q89R+E129V+K177L+R179E+Q254S+M284V truncated to 491 amino acids (using SEQ ID NO: 12 for numbering).
Alpha-Amylase X: Bacillus stearothermophilus alpha-amylase with the mutations: I181*+G182*+N193F truncated to 491 amino acids (using SEQ ID NO: 12 for numbering).
Glucoamylase Po: Mature part of the Penicillium oxalicum glucoamylase disclosed as SEQ ID NO: 2 in WO 2011/127802 and shown in SEQ ID NO: 13 herein.
Protease Pfu: Protease derived from Pyrococcus furiosus shown in SEQ ID NO: 5 herein.
Glucoamylase Po 498 (GA498): Variant of Penicillium oxalicum glucoamylase having the following mutations: K79V+P2N+P4S+P11F+T65A+Q327F (using SEQ ID NO: 13 for numbering).
Alpha-amylase blend A: Blend comprising Alpha-amylase AA369, glucoamylase GA498, and protease PfuS (dosing: 2.1 μg EP/g DS AA369, 4.5 μg EP/g DS GA498, 0.0385 μg EP/g DS PfuS, where EP is enzyme protein and DS is total dry solids)
Glucoamylase blend A: Blend comprising Talaromyces emersonii glucoamylase disclosed as SEQ ID NO: 34 in WO99/28448 and SEQ ID NO: 8 herein, Trametes cingulata glucoamylase disclosed as SEQ ID NO: 2 in WO 06/69289 and SEQ ID NO: 7, and Rhizomucor pusillus alpha-amylase with Aspergillus niger glucoamylase linker and starch binding domain (SBD) disclosed in SEQ ID NO: 6 herein having the following substitutions G128D+D143N using SEQ ID NO: 6 for numbering (activity ratio in AGU:AGU:FAU-F is about 29:8:1).
Metallo-protease from family M35 (AP025) from Thermoascus aurantiacus strain no. CGMCC 0670 was isolated from a soil sample collected on Jul. 21, 1998 in the Yunnan Province, Xishuangbanna, China. This protease was previously disclosed in WO 2003/048353 and included herein as SEQ ID NO: 1.

### Example 1: Cultivation of Thermoascus aurantiacus CGMCC No. 0670

Thermoascus aurantiacus CGMCC No. 0670 was grown at 45° C. for 60 hours in shake flasks with the CBH1 medium. The culture broth was harvested by centrifugation (7000 rpm for 20 minutes at 4° C.). A total of 1500 ml culture broth was obtained.

### Example 2: Purification of the Protease of Thermoascus aurantiacus CGMCC No. 0670

1500 ml supernatant from Example 2 was precipitated with ammonium sulfate (80% saturation) and re-dissolved in 40 ml 25 mM Tris-HCl, pH7.4 buffer. The resulting solution was ultra-filtrated with a 5K membrane to remove salts and change buffer to 25 mM Tris-HCl, pH7.4, following which it was filtered through a 0.45 μm filter. The final volume was 30 ml. The solution was applied to a 20 ml Q Sepharose FF column equilibrated in 25 mM Tris-HCl, pH7.4, and the proteins were eluted with a linear NaCl gradient (0-0.4M). Fractions from the column were analyzed for protease activity on AZCL-casein at pH 9.0, with or without SSI. Fractions with protease activity not inhibited by SSI were pooled. Then the pooled solution was applied to a Superdex-75 column equilibrated with 25 mM Tris-HCl, pH7.4, and the proteins were eluted with the same buffer. Protease-containing fractions were analyzed by SDS-PAGE and pure fractions were pooled.

The purity of the purified protease was checked by SDS-page and on an IEF gel. The sample contained only one protease which was designated AP025 and disclosed herein as amino acids 1 to 177 of SEQ ID NO: 1. The molecular weight is around 23 kDa and the pI is pH8.5.

### Example 3. Effect of Exo-Peptidase of Tripeptidylaminopeptidase from A. niger or T. reesei Combination with Endo-Protease from Thermoascus aurantiacus for Increasing Ethanol Titer in Simultaneous Saccharification and Fermentation Process

An industrial prepared liquefied mash using Alpha-amylase-X was used for the experiment. The dry solid determined by moisture balance (Mettler-Toledo) was about 30.8% DS and pH was adjusted to pH 5.0 following by supplemented with 3 ppm of penicillin and 400 ppm of urea. Simultaneous saccharification and fermentation (SSF) was performed via mini-scale fermentations. Approximately 5 g of the industrial liquefied corn mash was added to 15 ml tube vials. Each vial was dosed with 0.6 AGU/gDS of Glucoamylase blend A and appropriate amount of endo-protease (SEQ ID NO: 1) from Thermoascus aurantiacus belong to family M35 with or without exo-peptidase namely tripeptidylaminopeptidase (TPAP) belong to family S53 from Aspergillus niger (SEQ ID NO: 4) or Trichoderma reesei (SEQ ID NO: 3), respectively, as shown in table below followed by addition of 100 micro liters hydrated yeast per 5 g slurry. As control, glucoamylase and 400 ppm urea was added but no addition of endo-protease or exo-peptidase. Actual glucoamylase and protease dosages were based on the exact weight of corn slurry in each vial. Vials were incubated at 32° C. Three replicates were selected for 52-hour time point analysis. At each time point, fermentation was stopped by addition of 50 micro liters of 40% H₂SO₄, follow by centrifuging, and filtering through a 0.45 micrometer filter. Ethanol and oligosaccharides concentration were determined using HPLC.

| | Endo- | Exo- | Exo- |
|---|---|---|---|
| | protease | peptidase | peptidase |
| | from T. | from A. | from T. |
| | aurantiacus | niger | reesei |
| Treatments | (μg/g DS) | μg/gDS | μg/gDS |
| 1. Control | — | — | |
| 2. Endo-protease only | 2.5 | — | |
| 3. Endo-protease only | 5.0 | | |
| 4. Endo-protease + A. niger | 2.5 | 2.5 | |
| Tripeptidylaminopeptidase | | | |
| 5. Endo-protease + T. reesei | 2.5 | | 2.5 |
| Tripeptidylaminopeptidase | | | |

As shown in result tables below, combination of M35 family endo-protease with S53 family of TPAP exo-peptidase increased ethanol yield with statistically significant compared to control or endo-protease alone.
Ethanol yield at 52 hour of endo-protease without or with exo-peptidase.

| Treatments | Ethanol (g/l) |
|---|---|
| 1. Control | 122.21 |
| 2. Endo-protease only | 125.07 |
| 3. Endo-protease only | 126.10 |
| 4. Endo-protease + A. niger Tripeptidylaminopeptidase | 126.22 |
| 5. Endo-protease + T. reesei Tripeptidylaminopeptidase | 126.22 |

1. A process for producing a fermentation product from starch-containing material comprising:
a) saccharifying the starch-containing material at a temperature below the initial gelatinization temperature of said starch-containing material using a carbohydrate-source generating enzymes; and
b) fermenting using a fermenting organism; wherein
steps a) and/or b) is performed in the presence of an endo-protease and an exo-protease mixture, wherein the exo-protease makes up at least 5% (w/w) of the protease mixture on a total protease enzyme protein basis, and wherein the endo-protease is selected from a family M35 endo-protease and the exo-protease is selected from a family S53 exo-protease.

2. A process for producing a fermentation product from starch-containing material comprising the steps of:
(a) liquefying starch-containing material at a temperature above the initial gelatinization temperature of said starch-containing material in the presence of an alpha-amylase;
(b) saccharifying the liquefied material obtained in step (a) using a carbohydrate-source generating enzyme;
(c) fermenting using a fermenting organism;
wherein steps b) and/or c) is performed in the presence of an endo-protease and an exo-protease mixture, wherein the exo-protease makes up at least 5% (w/w) of the protease mixture on a total protease enzyme protein basis, and wherein the endo-protease is selected from a family M35 endo-protease and the exo-protease is selected from a family S53 exo-protease.

3. The process according to claim 1, wherein saccharification and fermentation is performed simultaneously.

4. The process according to claim 1, wherein the exo-protease makes up at least 10% (w/w) of the protease mixture on a total protease enzyme protein basis, such as at least 15%, at least 20%, at least 25%, at least 30%, at least 35%, at least 40%, at least 45%, at least 50%, at least 55%, at least 60%, at least 65%, at least 70%, particularly at least 75%, more particularly the exo-protease makes up from between 5 to 95% (w/w) on a total protease enzyme protein basis, particularly 10 to 80% (w/w), particularly 15 to 70% (w/w), more particularly 20 to 60% (w/w), and even more particularly 25 to 50% (w/w) of the protease mixture in the composition on a total protease enzyme protein basis.

5. The process according to claim 1, wherein the endo-protease and exo-protease is present in a ratio of 5:2 micro grams enzyme protein (EP)/g dry solids (DS), particularly 5:3, more particularly 5:4.

6. The process of claim 1, wherein the endo-protease is selected from the M35 family, more particularly M35 protease derived from Thermoascus aurantiacus, the mature polypeptide of which comprises amino acids 1-177 of SEQ ID NO: 1 or a polypeptide having at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% identity to the polypeptide of SEQ ID NO: 1.

7. The process according to claim 1, wherein the S53 exo-protease is derived from a strain of Aspergillus, Trichoderma, Thermoascus, or Thermomyces, particularly Aspergillus oryzae, Aspergillus niger, Trichoderma reesei, Thermoascus thermophilus, or Thermomyces lanuginosus.

8. The process according to claim 7, wherein the S53 exo-protease is selected from a serine protease having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 3; or from a serine protease having at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4.

9. The process of claim 1, wherein an alpha-amylase is present or added during saccharification and/or fermentation.

10. The process according to claim 9, wherein the alpha-amylase is an acid alpha-amylase, preferably an acid fungal alpha-amylase.

11. The process of claim 1, wherein the carbohydrate-source generating enzyme is selected from the group consisting of glucoamylase, alpha-glucosidase, maltogenic amylase, pullulanase, and beta-amylase.

12. The process of claim 1, wherein the fermentation product is an alcohol, preferably ethanol, especially fuel ethanol, potable ethanol and/or industrial ethanol.

13. A composition comprising a mixture of endo-protease and exo-protease, wherein the endo-protease is selected from a family M35 endo-protease and the exo-protease is selected from a family S53 exo-protease.


SEQUENCE LISTING (13 sequences)

SEQ ID NO: 1 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 2 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 3 - protein, 415 aa, Trichoderma reesei
QEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 4 - protein, 575 aa, Aspergillus niger
IVHEKLAAVPSGWHHVEDAGSDHQISLSIALARKNLDQLESKLKDLSTPGESQYGQWLDQEDVDTLFPVASDKAVINWLRSANITHISRQGSLVNFATTVDKVNKLLNATFAYYQSGSSQRLRTTEYSIPDDLVDSIDLISPTTFFGKEKTTAGLNQRAQKIDTHVAKRSNSSSCADVITLSCLKEMYNFGNYTPSASSGSKLGFGSFLNESASYSDLAKFEKLFNLPSQSFSVELVNGGVNDQNQSTASLTEADLDVELLVGVAHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNRTQFNPIFPATCPYVTAVGGTMSYAPEIAWEASSGGFSNYFERAWFQKEAVQNYLAHHITNETKQYYSQFANFSGRGFPDVAAHSFEPSYEVIFYGARYGSGGTSAACPLFSALVGMLNDARLRAGKSTLGFLNPLLYSKGYRALTDVTGGQSIGCNGIDPQNDETVAGAGIIPWAHWNATVGWDPVTGLGLPDFEKLRQLVLSL

SEQ ID NO: 5 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 6 - protein, 583 aa, Artificial
  Hybrid alpha-amylase
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 7 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 8 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 9 - protein, 555 aa, Pycnoporus sanguineus
QSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 10 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 11 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 12 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 13 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF